(19) United States
(12) Patent Application Publication
(10) Pub. No.: US 2007/0260056 A1
(43) Pub. Date: Nov. 8, 2007

(54) Process for Preparing Bicyclic Pyrazolyl

(76) Inventor: Thomas A. Brandt, North Stonington, CT (US)

Correspondence Address: PFIZER INC., PATENT DEPARTMENT, MS8260-1611, EASTERN POINT ROAD, GROTON, CT, 06340, US

(21) Appl. No.: 11/661,911

(22) PCT Filed: Sep. 15, 2005
(86) PCT No.: PCT/IB05/03027

§ 371 Date: Mar. 1, 2007

Related U.S. Application Data
(60) Provisional application No. 60/613,613, filed on Sep. 27, 2004.

(51) Int. Cl. C07D 498/04

(52) U.S. Cl. 540/490

(57) ABSTRACT

A process for preparing compounds of Formula (I) is described herein. The compounds have been shown to act as cannabinoid receptor ligands and are therefore useful in the treatment of disease linked to the mediation of the cannabinoid receptors in animals.

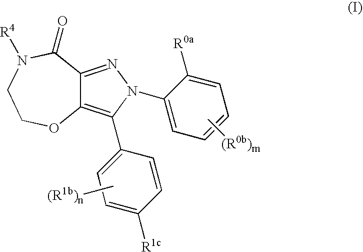

### FIELD OF THE INVENTION

The present invention relates to an improved synthetic process for preparing bicyclic pyrazolyl compounds, in particular, 3-(4-chloro-phenyl)-2-(2-chloro-phenyl)-7-(2,2-difluoro-propyl)-6,7-dihydro-2H,5H-4-oxa-1,2,7-triaza-azulen-8-one. The bicyclic pyrazolyl compounds have been found to be CB1 receptor antagonists and are therefore useful for treating diseases, conditions and/or disorders modulated by cannabinoid receptor antagonists.

### BACKGROUND

CB-1 antagonists have been shown to useful for the treatment of a variety of diseases, conditions and/or disorders including obesity, alcoholism, smoking cessation, Parkinson's disease, sexual dysfunctions, dementia, and so forth. Consequently, there exists a desire to develop compounds that antagonize the CB-1 receptor. US Publication No. 2005/0101592 (U.S. Provisional Patent Application Ser. No. 60/518,280 filed on Nov. 7, 2003) describes a series of bicyclic pyrazolyl and imidazolyl compounds that act as CB-1 antagonists. However, there exists a need to produce bicyclic pyrazolyl compounds, in particular 3-(4-chloro-phenyl)-2-(2-chloro-phenyl)-7-(2,2-difluoro-propyl)-6,7-dihydro-2H,5H-4-oxa-1,2,7-triaza-azulen-8-one, in a more efficient and cost effective means at larger scales of manufacture.

### SUMMARY

The present invention provides an improved process for preparing compounds of Formula (I): 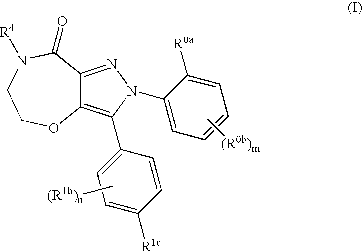
wherein

R^{0a}, R^{0b}, R^{1b}, and R^{1c} are each independently halo, (C₁-C₄)alkoxy, (C₁-C₄)alkyl, halo-substituted (C₁-C₄)alkyl, or cyano (preferably, R^{0a} is chloro, fluoro, or methyl; R^{0b} is chloro, fluoro, or hydrogen (i.e., m is 0); R^{1c} is chloro, fluoro, (C₁-C₄)alkyl, trifluoromethyl, (C₁-C₄)alkoxy, or cyano; and R^{1b} is hydrogen (i.e., n is 0));

n and m are each independently 0, 1 or 2 (preferably, n and m are 0 or 1, more preferably, n and m are both 0);

R⁴ is a chemical moiety selected from the group consisting of (C₁-C₈)alkyl, aryl, heteroaryl, aryl(C₁-C₄)alkyl, a 3- to 8-membered partially or fully saturated carbocyclic ring(s), heteroaryl(C₁-C₃)alkyl, 5-6 membered lactone, 5- to 6-membered lactam, and a 3- to 8-membered partially or fully saturated heterocycle, where said chemical moiety is optionally substituted with one or more substituents;

a pharmaceutically acceptable salt thereof, or a solvate or hydrate of the compound, or the salt.

Preferably, R⁴ is a chemical moiety selected from the group consisting of (C₁-C₈)alkyl, aryl(C₁-C₄)alkyl, 3- to 8-membered partially or fully saturated carbocyclic ring(s), and 3- to 8-membered partially or fully saturated heterocycle, where said chemical moiety is optionally substituted with one or more substituents. More preferably, R⁴ is (C₁-C₈)alkyl, halo-substituted (C₁-C₈)alkyl (preferably, fluoro-substituted (C₁-C₈)alkyl), cyclopentyl, cyclohexyl, piperidin-1-yl, pyrrolidin-1-yl, or morpholin-1-yl.

Most preferably, the compound of Formula (I) is 3-(4-chloro-phenyl)-2-(2-chloro-phenyl)-7-(2,2-difluoro-propyl)-6,7-dihydro-2H,5H-4-oxa-1,2,7-triaza-azulen-8-one (e.g., R^{0a} and R^{1c} are both chloro; n and m are both 0; and R⁴ is 2,2-difluoro-n-propyl).

The process for preparing the compounds of Formula (I) described above comprises the steps of:

(1) protecting the hydroxy group of a compound of Formula (1a) with a hydroxy-protecting group to form a hydroxy-protected compound of Formula (1b) 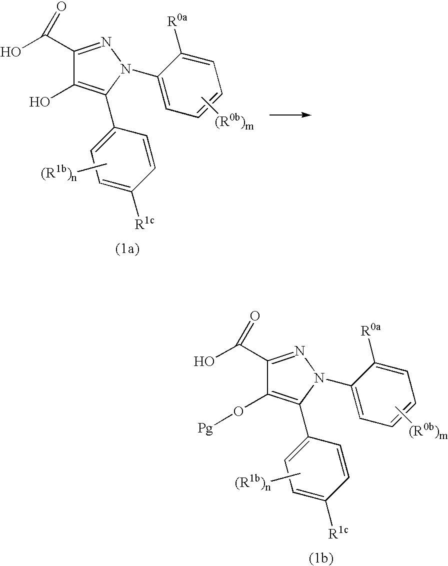
where R^{0a}, R^{0b}, R^{1b}, R^{1c}, m, and n are as defined above for the compound of Formula (I) and Pg is a hydroxy-protecting group;

(2) reacting the hydroxy-protected compound of Formula (1b) with a compound of Formula (1c) to form a compound of Formula (1d) 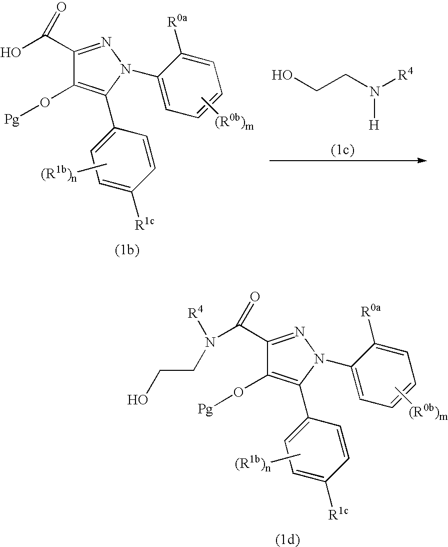
where R^{0a}, R^{0b}, R^{1b}, R^{1c}, m, n and R⁴ are as defined above for the compound of Formula (I) and Pg is a hydroxy-protecting group;

(3) converting the hydroxy group of the compound of Formula (1d) to a leaving group to produce a compound of Formula (1e) 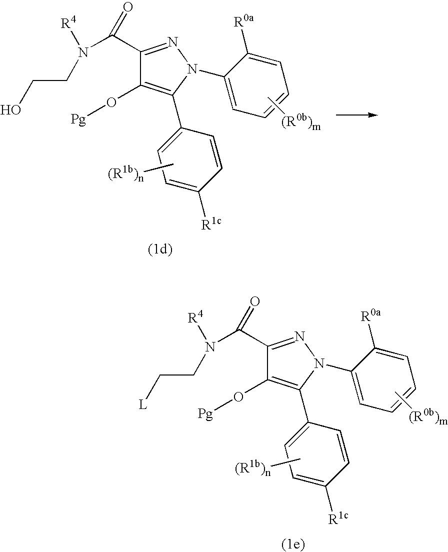
where R^{0a}, R^{0b}, R^{1b}, R^{1c}, m, n and R⁴ are as defined above for the compound of Formula (I), Pg is a hydroxy-protecting group, and L is a leaving group (e.g., halo, mesylate, tosylate, or any group capable of being displaced by an oxygen anion);

(4) removing said hydroxy-protecting group and cyclizing the compound of Formula (1e) to form the compound of Formula (I); and

(5) isolating the compound of Formula (I), or a pharmaceutically acceptable salt thereof, or a solvate or hydrate of the compound or salt.

An advantage of this process is that the compound of Formula (1a) can be converted to the compound of Formula (1e) without isolating the compound of Formula (1b) or the compound of Formula (1d).

The process described above may further comprise the following step

wherein the compound of Formula (1a) is prepared by a method comprising the steps of

(i) reacting a compound of Formula (2a) with a dialkyl oxalate in the presence of an alkali metal base (e.g., an alkali metal amide of a sterically hindered secondary amine (lithium bis(trimethylsilyl)-amide, lithium diisopropylamide, and lithium 2,2,6,6-tetramethylpiperidine), an alkali metal hydride (e.g., lithium hydride, sodium hydride, potassium hydride) or an alkali metal alkoxide (e.g., sodium ethoxide and sodium methoxide)) to form a compound of Formula (2b) 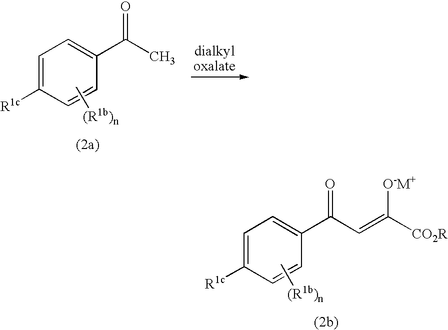
where R^{1b}, R^{1c} and n are as described above for the compound of Formula (I), M is an alkali metal (e.g., lithium, sodium or potassium) and R is a (C₁-C₆)alkyl group;

(ii) reacting the compound of Formula (2b) with a compound of Formula (2c) followed by treatment with an alkali metal hydroxide to form a compound of Formula (2d) 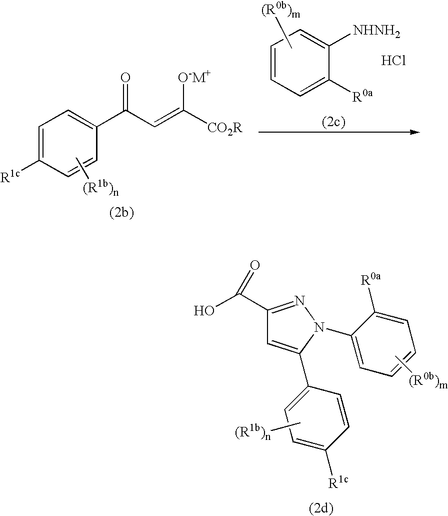
where R is a (C₁-C₆)alkyl group, M is as defined above, and R^{0a}, R^{0b}, R^{1b}, R^{1c}, n and m are as defined above for the compound of Formula (I); and

(iii) reacting the compound of Formula (2d) with a trialkylborate in the presence of an alkyl lithium followed by treating with basic hydrogen peroxide to produce the compound of Formula (1a). An example of a basic hydrogen peroxide is NaOOH which is formed by mixing aqueous sodium hydroxide with aqueous hydrogen peroxide.

In an alternative process, the compound of Formula (1a) can be prepared by a method comprising the step of

(i) hydrolyzing a compound of Formula (3d) to form the compound of Formula (1a) 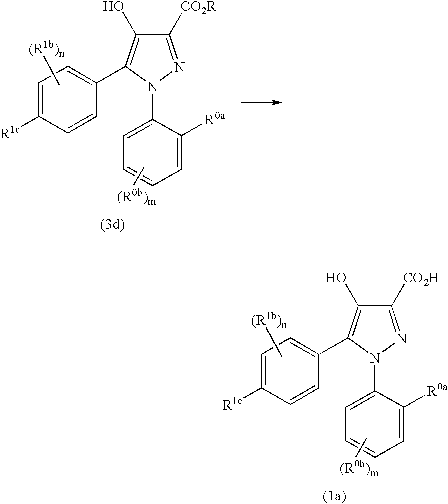
where R^{0a}, R^{0b}, R^{1b}, R^{1c}, n and m are as defined above for the compound of Formula (I); and R is (C₁-C₆)alkyl.

In a preferred embodiment, the process is used to produce 3-(4-chloro-phenyl)-2-(2-chloro-phenyl)-7-(2,2-difluoro-propyl)-6,7-dihydro-2H,5H-4-oxa-1,2,7-triaza-azulen-8-one, or a solvate or hydrate thereof which comprises the steps of:

(1) protecting the hydroxy group of a compound of Formula (1a-1) with an acetyl group to form a compound of Formula (1b-1);

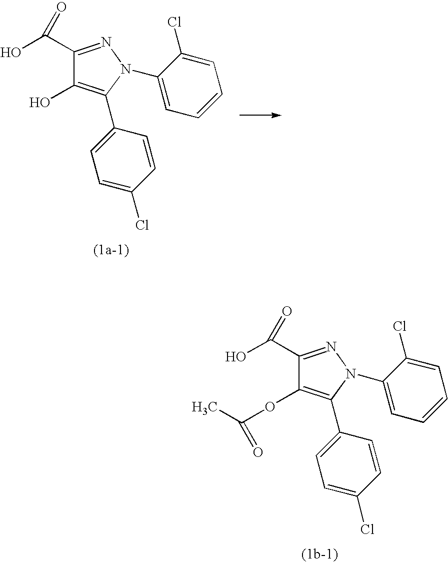

(2) reacting said compound of Formula (1b-1) with a compound of Formula (1c-1) to form a compound of Formula (1d-1)

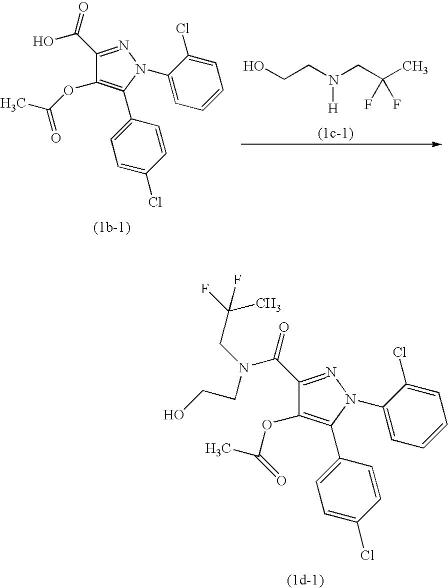

(3) converting the hydroxy group of said compound of Formula (1d-1) to a chloro group to produce a compound of Formula (1e-1)

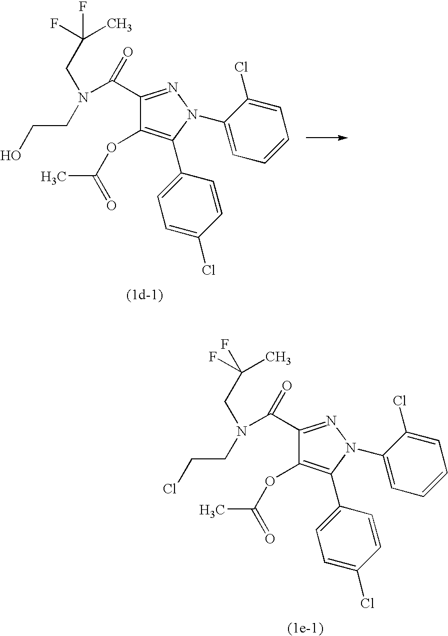

(4) removing said acetyl group and cyclizing said compound of Formula (1e-1) to form 3-(4-chloro-phenyl)-2-(2-chloro-phenyl)-7-(2,2-difluoro-propyl)-6,7-dihydro-2H,5H-4-oxa-1,2,7-triaza-azulen-8-one; and

(5) isolating said 3-(4-chloro-phenyl)-2-(2-chloro-phenyl)-7-(2,2-difluoro-propyl)-6,7-dihydro-2H,5H-4-oxa-1,2,7-triaza-azulen-8-one, or a solvate or hydrate thereof.

An advantage of this synthetic route is that the compound of Formula (1a-1) can be converted to the compound of Formula (1e-1) without isolating the compound of Formula (1b-1), or the compound of Formula (1d-1).

The compound of Formula (1a-1) may be prepared by treating a compound of Formula (2d-1) with an alkyl lithium (preferably, hexyllithium) and then reacting with a trialkylborate (preferably trimethylborate) followed by treating with basic hydrogen peroxide to produce the compound of Formula (1a-1).

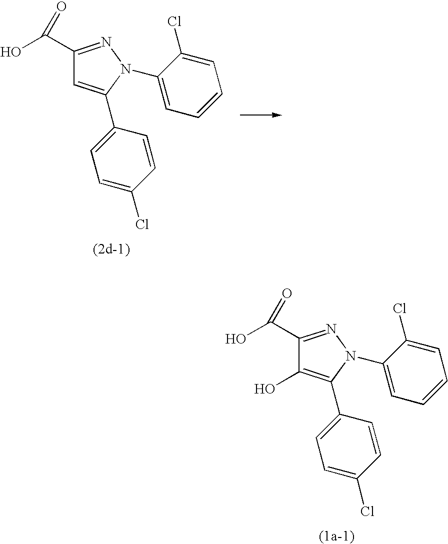

In another embodiment of the present invention, an intermediate having the Formula (1d) is provided. 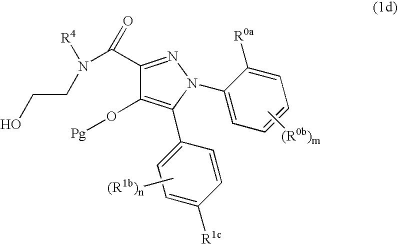
wherein

Pg is a hydroxy-protecting group;

R^{0a}, R^{0b}, R^{1b}, and R^{1c} are each independently halo, (C₁-C₄)alkoxy, (C₁-C₄)alkyl, halo-substituted (C₁-C₄)alkyl, or cyano;

n and m are each independently 0, 1 or 2; and

R⁴ is a chemical moiety selected from the group consisting of (C₁-C₈)alkyl, aryl, heteroaryl, aryl(C₁-C₄)alkyl, a 3- to 8-membered partially or fully saturated carbocyclic ring(s), heteroaryl(C₁-C₃)alkyl, 5-6 membered lactone, 5- to 6-membered lactam, and a 3- to 8-membered partially or fully saturated heterocycle, where said chemical moiety is optionally substituted with one or more substituents. Preferably, Pg is acetyl; R^{0a} and R^{1c} are both chloro; m and n are both 0; and R⁴ is 2,2-difluoro-n-propyl.

In yet another embodiment of the present invention, an intermediate having the Formula (1e) is provided. 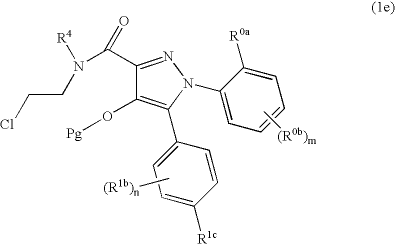
wherein

Pg is a hydroxy-protecting group;

R^{0a}, R^{0b}, R^{1b}, and R^{1c} are each independently halo, (C₁-C₄)alkoxy, (C₁-C₄)alkyl, halo-substituted (C₁-C₄)alkyl, or cyano;

n and m are each independently 0, 1 or 2; and

R⁴ is a chemical moiety selected from the group consisting of (C₁-C₈)alkyl, aryl, heteroaryl, aryl(C₁-C₄)alkyl, a 3- to 8-membered partially or fully saturated carbocyclic ring(s), heteroaryl(C₁-C₃)alkyl, 5-6 membered lactone, 5- to 6-membered lactam, and a 3- to 8-membered partially or fully saturated heterocycle, where said chemical moiety is optionally substituted with one or more substituents. Preferably, Pg is acetyl; R^{0a} and R^{1c} are both chloro; m and n are both 0; and R⁴ is 2,2-difluoro-n-propyl.

Some of the compounds prepared by the processes described herein may exist as rotamers. For example, at least two major rotameric species have been observed by NMR for intermediates 1d-1, 1e-1, and I-1f (deprotected 1e-1). In addition, tautomeric forms of the compounds are also within the scope of the present invention.

### Definitions

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy, acyl (e.g., alkanoyl), alkylamino, dialkylamino, and alkylthio group have the same definition as above. Unless specified otherwise, “alkyl” is a general designation for a (C₁-C₆)alkyl. When indicated as being “optionally substituted”, the alkane radical or alkyl moiety may be unsubstituted or substituted with one or more substituents (generally, one to three substituents except in the case of halogen substituents such as perchloro or perfluoroalkyls) independently selected from the group of substituents listed below in the definition for “substituted.” “Halo-substituted alkyl” refers to an alkyl group substituted with one or more halogen atoms (e.g., “fluoro-substituted alkyl” refers to fluoromethyl, difluoromethyl, trifluoromethyl, 1-fluoroethyl, 2-fluoroethyl, 1,1-difluoroethyl, 1,2-difluoroethyl, 2,2-difluoroethyl, 1,1,1-trifluoroethyl, 2,2,2-trifluoroethyl, 1,1,2-trifluoroethyl, 1,2,2-trifluoroethyl, 1,2,2,2-tetrafluoroethyl, 1,1,2,2-tetrafluoroethyl, 1,1,1,2-tetrafluoroethyl, 1,1,2,2,2-pentafluoroethyl, 1,1,1,2,2-pentafluoroethyl, perfluoroethyl, etc.). Preferred halo-substituted alkyls are the chloro- and fluoro-substituted alkyls, more preferably, fluoro-substituted alkyls. When substituted, the alkane radicals or alkyl moieties are preferably fluoro substituents (as described above), or 1 or 2 substituents independently selected from (C₁-C₃)alkyl, (C₃-C₆)cycloalkyl, (C₂-C₃)alkenyl, aryl, heteroaryl, 3- to 6-membered heterocycle, chloro, cyano, hydroxy, (C₁-C₃)alkoxy, aryloxy, amino, (C₁-C₆)alkyl amino, di-(C₁-C₄)alkyl amino, aminocarboxylate (i.e., (C₁-C₃)alkyl-O—C(O)—NH—), hydroxy(C₂-C₃)alkylamino, or keto (oxo), and more preferably, 1 to 3 fluoro groups, or 1 substituent selected from (C₁-C₃)alkyl, (C₃-C₆)cycloalkyl, (C₆)aryl, 6-membered-heteroaryl, 3- to 6-membered heterocycle, (C₁-C₃)alkoxy, (C₁-C₄)alkyl amino or di-(C₁-C₂)alkyl amino.

The terms “partially or fully saturated carbocyclic ring” (also referred to as “partially or fully saturated cycloalkyl”) refers to nonaromatic rings that are either partially or fully hydrogenated and may exist as a single ring, bicyclic ring or a spiral ring. Unless specified otherwise, the carbocyclic ring is generally a 3- to 8-membered ring. For example, partially or fully saturated carbocyclic rings (or cycloalkyl) include groups such as cyclopropyl, cyclopropenyl, cyclobutyl, cyclobutenyl, cyclopentyl, cyclpentenyl, cyclopentadienyl, cyclohexyl, cyclohexenyl, cyclohexadienyl, norbornyl (bicyclo[2.2.1]heptyl), norbornenyl, bicyclo[2.2.2]octyl, and the like. When designated as being “optionally substituted”, the partially saturated or fully saturated cycloalkyl group may be unsubstituted or substituted with one or more substituents (typically, one to three substituents) independently selected from the group of substituents listed below in the definition for “substituted.” A substituted carbocyclic ring also includes groups wherein the carbocyclic ring is fused to a phenyl ring (e.g., indanyl). The carbocyclic group may be attached to the chemical entity or moiety by any one of the carbon atoms within the carbocyclic ring system. When substituted, the carbocyclic group is preferably substituted with 1 or 2 substituents independently selected from (C₁-C₃)alkyl, (C₂-C₃)alkenyl, (C₁-C₆)alkylidenyl, aryl, heteroaryl, 3- to 6-membered heterocycle, chloro, fluoro, cyano, hydroxy, (C₁-C₃)alkoxy, aryloxy, amino, (C₁-C₆)alkyl amino, di-(C₁-C₄)alkyl amino, aminocarboxylate (i.e., (C₁-C₃)alkyl-O—C(O)—NH—), hydroxy(C₂-C₃)alkylamino, or keto (oxo), and more preferably 1 or 2 from substituents independently selected from (C₁-C₂)alkyl, 3- to 6-membered heterocycle, fluoro, (C₁-C₃)alkoxy, (C₁-C₄)alkyl amino or di-(C₁-C₂)alkyl amino. Similarly, any cycloalkyl portion of a group (e.g., cycloalkylalkyl, cycloalkylamino, etc.) has the same definition as above.

The term “partially saturated or fully saturated heterocyclic ring” (also referred to as “partially saturated or fully saturated heterocycle”) refers to nonaromatic rings that are either partially or fully hydrogenated and may exist as a single ring, bicyclic ring or a spiral ring. Unless specified otherwise, the heterocyclic ring is generally a 3- to 6-membered ring containing 1 to 3 heteroatoms (preferably 1 or 2 heteroatoms) independently selected from sulfur, oxygen and/or nitrogen. Partially saturated or fully saturated heterocyclic rings include groups such as epoxy, aziridinyl, tetrahydrofuranyl, dihydrofuranyl, dihydropyridinyl, pyrrolidinyl, N-methylpyrrolidinyl, imidazolidinyl, imidazolinyl, piperidinyl, piperazinyl, pyrazolidinyl, 2H-pyranyl, 4H-pyranyl, 2H-chromenyl, oxazinyl, morpholino, thiomorpholino, tetrahydrothienyl, tetrahydrothienyl 1,1-dioxide, and the like. When indicated as being “optionally substituted”, the partially saturated or fully saturated heterocycle group may be unsubstituted or substituted with one or more substituents (typically, one to three substituents) independently selected from the group of substituents listed below in the definition for “substituted.” A substituted heterocyclic ring includes groups wherein the heterocyclic ring is fused to an aryl or heteroaryl ring (e.g., 2,3-dihydrobenzofuranyl, 2,3-dihydroindolyl, 2,3-dihydrobenzothiophenyl, 2,3-dihydrobenzothiazolyl, etc.). When substituted, the heterocycle group is preferably substituted with 1 or 2 substituents independently selected from (C₁-C₃)alkyl, (C₃-C₆)cycloalkyl, (C₂-C₄)alkenyl, aryl, heteroaryl, 3- to 6-membered heterocycle, chloro, fluoro, cyano, hydroxy, (C₁-C₃)alkoxy, aryloxy, amino, (C₁-C₆)alkyl amino, di-(C₁-C₃)alkyl amino, aminocarboxylate (i.e., (C₁-C₃)alkyl-O—C(O)—NH—), or keto (oxo), and more preferably with 1 or 2 substituents independently selected from (C₁-C₃)alkyl, (C₃-C₆)cycloalkyl, (C₆)aryl, 6-membered-heteroaryl, 3- to 6-membered heterocycle, or fluoro. The heterocyclic group may be attached to the chemical entity or moiety by any one of the ring atoms within the heterocyclic ring system. Similarly, any heterocycle portion of a group (e.g., heterocycle-substituted alkyl, heterocycle carbonyl, etc.) has the same definition as above.

The term “aryl” or “aromatic carbocyclic ring” refers to aromatic moieties having a single (e.g., phenyl) or a fused ring system (e.g., naphthalene, anthracene, phenanthrene, etc.). A typical aryl group is a 6- to 10-membered aromatic carbocyclic ring(s). When indicated as being “optionally substituted”, the aryl groups may be unsubstituted or substituted with one or more substituents (preferably no more than three substituents) independently selected from the group of substituents listed below in the definition for “substituted.” Substituted aryl groups include a chain of aromatic moieties (e.g., biphenyl, terphenyl, phenylnaphthalyl, etc.). When substituted, the aromatic moieties are preferably substituted with 1 or 2 substituents independently selected from (C₁-C₄)alkyl, (C₂-C₃)alkenyl, aryl, heteroaryl, 3- to 6-membered heterocycle, bromo, chloro, fluoro, iodo, cyano, hydroxy, (C₁-C₄)alkoxy, aryloxy, amino, (C₁-C₆)alkyl amino, di-(C₁-C₃)alkyl amino, or aminocarboxylate (i.e., (C₁-C₃)alkyl-O—C(O)—NH—), and more preferably, 1 or 2 substituents independently selected from (C₁-C₄)alkyl, chloro, fluoro, cyano, hydroxy, or (C₁-C₄)alkoxy. The aryl group may be attached to the chemical entity or moiety by any one of the carbon atoms within the aromatic ring system. Similarly, the aryl portion (i.e., aromatic moiety) of an aroyl or aroyloxy (i.e., (aryl)-C(O)—O—) has the same definition as above.

The term “heteroaryl” or “heteroaromatic ring” refers to aromatic moieties containing at least one heteratom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, imidazolyl, tetrazolyl, triazinyl, pyrimidyl, pyrazinyl, thiazolyl, purinyl, benzimidazolyl, quinolinyl, isoquinolinyl, benzothiophenyl, benzoxazolyl, etc.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. When indicated as being “optionally substituted”, the heteroaryl groups may be unsubstituted or substituted with one or more substituents (preferably no more than three substituents) independently selected from the group of substituents listed below in the definition for “substituted.” When substituted, the heteroaromatic moieties are preferably substituted with 1 or 2 substituents independently selected from (C₁-C₄)alkyl, (C₂-C₃)alkenyl, aryl, heteroaryl, 3- to 6-membered heterocycle, bromo, chloro, fluoro, iodo, cyano, hydroxy, (C₁-C₄)alkoxy, aryloxy, amino, (C₁-C₆)alkyl amino, di-(C₁-C₃)alkyl amino, or aminocarboxylate (i.e., (C₁-C₃)alkyl-O—C(O)—NH—), and more preferably, 1 or 2 substituents independently selected from (C₁-C₄)alkyl, chloro, fluoro, cyano, hydroxy, (C₁-C₄)alkoxy, (C₁-C₄)alkyl amino or di-(C₁-C₂)alkyl amino. The heteroaryl group may be attached to the chemical entity or moiety by any one of the atoms within the aromatic ring system (e.g., imidazol-1-yl, imidazol-2-yl, imidazol-4-yl, imidazol-5-yl, pyrid-2-yl, pyrid-3-yl, pyrid-4-yl, pyrid-5-yl, or pyrid-6-yl). Similarly, the heteroaryl portion (i.e., heteroaromatic moiety) of a heteroaroyl or heteroaroyloxy (i.e., (heteroaryl)-C(O)—O—) has the same definition as above.

The term “substituted” specifically envisions and allows for one or more substitutions that are common in the art. However, it is generally understood by those skilled in the art that the substituents should be selected so as to not adversely affect the pharmacological characteristics of the compound or adversely interfere with the use of the medicament. Suitable substituents for any of the groups defined above include (C₁-C₆)alkyl, (C₃-C₇)cycloalkyl, (C₂-C₆)alkenyl, (C₁-C₆)alkylidenyl, aryl, heteroaryl, 3- to 6-membered heterocycle, halo (e.g., chloro, bromo, iodo and fluoro), cyano, hydroxy, (C₁-C₆)alkoxy, aryloxy, sulfhydryl (mercapto), (C₁-C₆)alkylthio, arylthio, amino, mono- or di-(C₁-C₆)alkyl amino, quaternary ammonium salts, amino(C₁-C₆)alkoxy, aminocarboxylate (i.e., (C₁-C₆)alkyl-O—C(O)—NH—), hydroxy(C₂-C₆)alkylamino, amino(C₁-C₆)alkylthio, cyanoamino, nitro, (C₁-C₆)carbamyl, keto (oxo), acyl, (C₁-C₆)alkyl-CO₂—, glycolyl, glycyl, hydrazino, guanyl, sulfamyl, sulfonyl, sulfinyl, thio(C₁-C₆)alkyl-C(O)—, thio(C₁-C₆)alkyl-CO₂—, and combinations thereof. In the case of substituted combinations, such as “substituted aryl(C₁-C₆)alkyl”, either the aryl or the alkyl group may be substituted, or both the aryl and the alkyl groups may be substituted with one or more substituents (typically, one to three substituents except in the case of perhalo substitutions). An aryl or heteroaryl substituted carbocyclic or heterocyclic group may be a fused ring (e.g., indanyl, dihydrobenzofuranyl, dihydroindolyl, etc.).

The term “solvate” refers to a molecular complex of a compound represented by Formula (I) or (II) (including prodrugs and pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The phrase “pharmaceutically acceptable” indicates that the substance or composition must be compatible chemically and/or toxicologically, with the other ingredients comprising a formulation, and/or the mammal being treated therewith.

### DETAILED DESCRIPTION

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

In the preparation of the bicyclic pyrazolyl compounds, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see Theodora W. Greene and Peter G. M. Wuts, Protective Groups in Organic Synthesis, John Wiley & Sons, New York, 2002.

Scheme I below summarizes the process of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives.

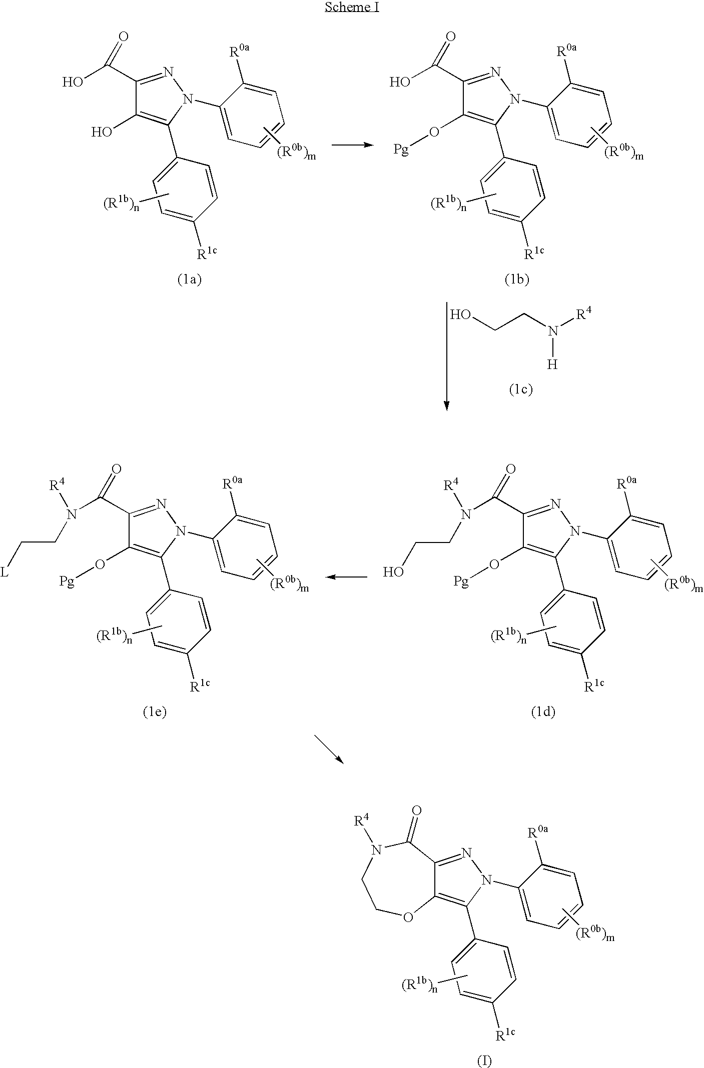

The compound of Formula (1a) can be prepared by either of the procedures outlined in Schemes II or III below. The hydroxy group on the pyrazole ring is protected with a hydroxy-protecting group before reacting with the desired hydroxyalkylamine compound (1c). Any hydroxy-protecting group can be used that is known in the art; however, an acetyl protecting group is preferred. For example, when an acetyl protecting group is used, a base (e.g., N,N-diisopropylethylamine) may be added slowly to a suspension of intermediate (1a) in a polar solvent (e.g., methylene chloride) at or slightly below room temperature followed by the addition of acetic anhydride. The carboxylic acid group on the pyrazole ring is then condensed with the desired hydroxyalkylamine compound (1c) to form an amide linkage. Standard amidation procedures well-known to those skilled in the art can be used. For example, compound of Formula (1b) can be treated with 2-chloro-4,6-dimethoxy-1,3,5-triazine followed by the addition of 4-methylmorpholine below room temperature and then slowly warmed to ambient temperature. The complex formed is then reacted with the desired hydroxyalkylamine compound (1c) to form the amide (1d) at a temperature between about 20° C. and about 25° C. The hydroxy group on the alkyl amine is converted to a leaving group (e.g., halo, mesylate, tosylate or any group capable of being displaced with the oxygen anion in the following cyclization reaction). When the leaving group is chloro, then the amide (1d) can be treated with a chlorinating agent (e.g., methanesulfonyl chloride in the presence of a base (e.g., N,N-diisopropylethylamine)) at a temperature of about 0° C. and then allowed to warm slowly to ambient temperature. Finally, the intermediate (1e) can be cyclized to the desired compound of Formula (I) (e.g., treatment with cesium carbonate at a temperature between about 20° C. and 30° C.). The advantage of this synthetic route is that the intermediate (1a) can be converted to the chloro intermediate (1e) in two steps without isolating any of the intervening intermediates (1b) or (1d).

An overview of ortho-metallation chemistry (i.e., conversion of intermediates 2d to 1a) may be found in Snieckus, V., Chem. Rev. (1990) 90, 879; Examples of ortho-metallation of aryl carboxylic acids can be found in Mortier, J., Moyroud, J, J. Org. Chem. (1994) 59, 4042 and Bennetau, B., Mortier, J., Moyroud, J., Guesnet, J., J. Chem. Soc. Perkin Trans. 1 (1995) 10, 1265. An example of an aryl-metal species being reacted with a trialkylborate followed by treatment with basic hydrogen peroxide to give a phenol can be found in Hawthorne, M., J. Org. Chem. (1957) 22, 1001. General procedures for using 4-methylmorpholine/2-chloro-4,6-dimethoxytriazine to make amides (i.e., conversion of intermediates 1b to 1d) may be found in Kaminski, Z. J., Synthesis (1987) 917; Kaminski, Z. J., Paneth, P., Rudzinski, J., J. Org. Chem. (1998) 63, 4248; and Garrett, C. E., Jiang, X., Prasad, K., Repic, O., Tetrahedron Letters (2002) 43, 4161. General procedures for deacetylation (i.e., conversion of intermediates 1e to 1f) may be found in Rapoport, H., Plattner, J. J., Gless, R. D., J. Am. Chem. Soc. (1972) 94, 8613

For a detailed preparation using the procedures described above, see the Example section below.

The hydroxy intermediate (1a) can be synthesized using the procedures described in Scheme II below.

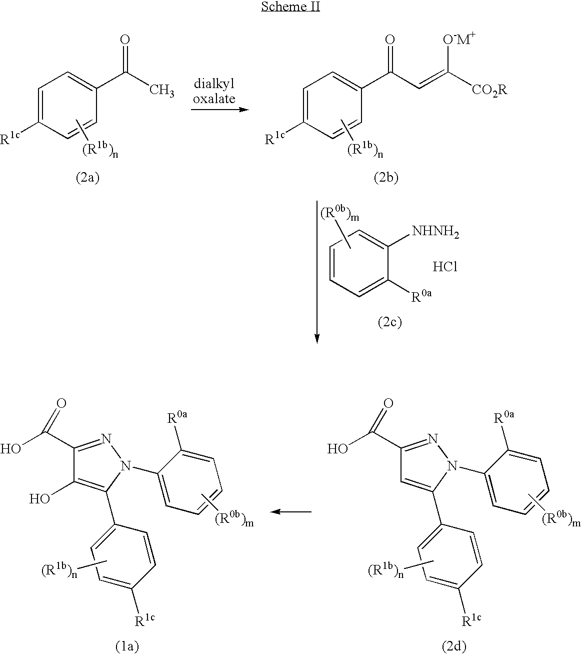

The desired starting material (2a) may be purchased from a variety of chemical suppliers or prepared using standard chemical preparations as described in standard chemical synthesis books (e.g., Beilstein). The pyrazole ring may be built by first reacting the desired compound of Formula (2a) with dialkyloxalate (e.g., dimethyloxalate or diethyloxalate) in the presence of a strong base (e.g., lithium bis(trimethylsilyl)amide) in a aprotic solvent (e.g., tert-butyl methyl ether and tetrahydrofuran). The resultant enol (2b) may then be reacted with the desired hydrazine salt (2c) in a polar solvent (e.g., ethanol) followed by treatment with a strong base (e.g., alkali metal hydroxide). A hydroxy group may then be attached to the pyrazole ring by treating the compound of Formula (2d) with an alkyl lithium (e.g., hexyllithium, n-butyllithium, sec-butyllithium and tert-butyllithium) and trialkylborate (e.g., trimethylborate, triethylborate and triisopropylborate) followed by treatment with basic hydrogen peroxide.

Alternatively, the hydroxy intermediate (1a) can be prepared using the synthetic steps outlined in Scheme III below. 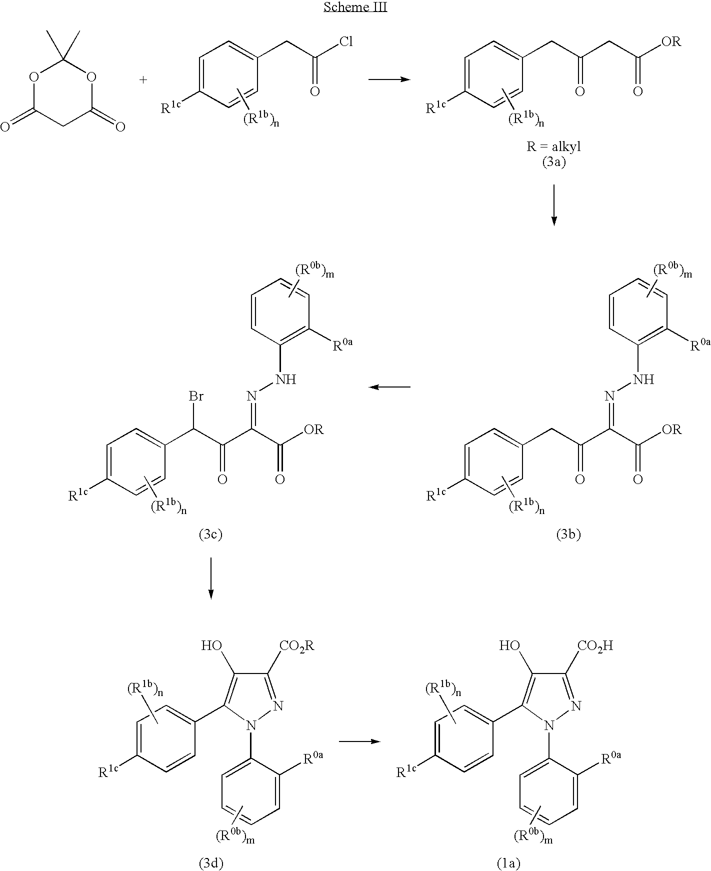
The keto ester intermediate (3a) can be prepared by condensing the desired acid chloride with 2,2-dimethyl-[1,3]dioxane-4,6-dione in the presence of a base (e.g., pyridine) in an aprotic solvent (e.g., methylene chloride) followed by heating at an elevated temperature in a protic solvent (e.g., ethanol). The hydrazono intermediate (3b) can then be prepared by treating the keto ester (3a) with the desired amine in the presence of sodium nitrate in an acidic medium (e.g., aqueous acetic acid). The bromo group may then be introduced using standard bromination procedures well-known to those skilled in the art. For example, intermediate (3b) can be treated with copper (II) bromide in an aprotic solvent (e.g., ethyl acetate and chloroform) at an elevated temperature. Cyclization of the bromo intermediate (3c) may then be accomplished by heating in a polar solvent (e.g., methanol) in the presence of sodium acetate. The hydroxy ester intermediate (3d) can then be hydrolyzed to the corresponding hydroxy carboxylic acid (1a) using conventional hydrolysis processes well-known to those skilled in the art. For example, the ester (3d) can be treated with a metal hydroxide (e.g., potassium hydroxide) in the presence of an aqueous protic solvent (e.g., methanol). For an example of a detailed preparation using the procedures described above, see the Example section below.

Conventional methods and/or techniques of separation and purification known to one of ordinary skill in the art can be used to isolate the compounds of the present invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography), recrystallization, and differential (i.e., liquid-liquid) extraction techniques.

The compounds may be isolated and used per se or in the form of its pharmaceutically acceptable salt, solvate and/or hydrate. The term “salts” refers to inorganic and organic salts of a compound of the present invention. These salts can be prepared in situ during the final isolation and purification of a compound, or by separately reacting the compound with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

The compounds may contain asymmetric or chiral centers, and, therefore, exist in different stereoisomeric forms. It is intended that all stereoisomeric forms of the compounds as well as mixtures thereof, including racemic mixtures, form part of the present invention. In addition, the present invention embraces all geometric and positional isomers. For example, if a compound incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

The compounds may exist in unsolvated as well as solvated forms with pharmaceutically acceptable solvents such as water, ethanol, and the like, and it is intended that the invention embrace both solvated and unsolvated forms.

It is also possible that the intermediates and compounds may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

The present invention also embraces isotopically-labeled compounds (including intermediates) which are identical to those recited herein, but for the fact that one or more atoms are replaced by an atom having an atomic mass or mass number different from the atomic mass or mass number usually found in nature. Examples of isotopes that can be incorporated into the intermediates or compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

The preparation of certain isotopically-labeled compounds (e.g., those labeled with ³H and ¹⁴C) is useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Compounds made by the process of the present invention are useful for treating diseases, conditions and disorders modulated by cannabinoid receptor antagonists.

Preliminary investigations have indicated that the following diseases, conditions, and/or disorders are modulated by cannabinoid receptor antagonists: eating disorders (e.g., binge eating disorder, anorexia, and bulimia), weight loss or control (e.g., reduction in calorie or food intake, and/or appetite suppression), obesity, depression, atypical depression, bipolar disorders, psychoses, schizophrenia, behavioral addictions, suppression of reward-related behaviors (e.g., conditioned place avoidance, such as suppression of cocaine- and morphine-induced conditioned place preference), substance abuse, addictive disorders, impulsivity, alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), dementia (including memory loss, Alzheimer's disease, dementia of aging, vascular dementia, mild cognitive impairment, age-related cognitive decline, and mild neurocognitive disorder), sexual dysfunction in males (e.g., erectile difficulty), seizure disorders, epilepsy, inflammation, gastrointestinal disorders (e.g., dysfunction of gastrointestinal motility or intestinal propulsion), attention deficit disorder (ADD including attention deficit hyperactivity disorder (ADHD)), Parkinson's disease, and type II diabetes.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, reagents, solvents and starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (CI) were obtained on a Hewlett-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. In some cases only representative ¹H NMR peaks are given. MS peaks are reported for all examples. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) under low nitrogen pressure.

The following section provides representative examples of useful starting materials and/or intermediates that may be used in the process of the present invention.

### Starting Materials and/or Intermediates

### Preparation of 4-(4-chlorophenyl)-2-hydroxy-4-oxo-but-2-enoic acid ethyl ester lithium salt (I-2b)

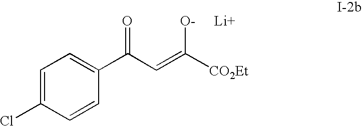

Lithium bis(trimethylsilyl)-amide (149 ml: 1.0 M in tetrahydrofuran, 149 mmol) was added to tert-butyl methyl ether (350 ml) at room temperature. The resulting solution was then cooled to −75° C. 1-(4-Chlorophenyl)ethanone (23.28 g, 150.6 mmoles) was added as a solution in 23 ml of tert-butyl methyl ether over 3 minutes while keeping the internal temperature less than −70° C. The reaction solution was allowed to stir for 1 hour at −75° C., then diethyl oxalate (22.0 g, 150 mmol) was added neat over 5 minutes while keeping the internal temperature less than −70° C. The clear dark orange reaction solution was then warmed to room temperature over 4 hours. (The product began to precipitate at −3° C.) The reaction was allowed to stir for 15 hours at room temperature, followed by isolation of the precipitated product by filtration. The filtercake was washed with 100 ml of room temperature tert-butyl methyl ether and then dried at 60° C. in vacuo for 1 hour to give 4-(4-chlorophenyl)-2-hydroxy-4-oxo-but-2-enoic acid ethyl ester lithium salt I-2b (36.72 g, 94%) as a powdery yellow solid.

¹H-NMR (DMSO-d₆) δ 7.80 (d, 1.94H, J=8.7 Hz), 7.66 (d, 0.06H, J=8.7 Hz), 7.43 (d, 1.94H, J=8.7 Hz), 7.31 (d, 0.06H, J=8.3 Hz), 6.37 (s, 0.97H), 5.22 (s, 0.03H), 4.10 (q, 1.94H, J=7.05 Hz), 4.00 (q, 0.06H, J=7.05 Hz), 1.20 (t, 2.91H, J=7.05 Hz), 1.15 (t, 0.09H, J=7.05 Hz). Shows a 97:3 mixture of geometric isomers. Mass Spec (ESI): M+1=255.2 (mass of neutral compound)

### Preparation of 1-(2-chlorophenyl)-5-(4-chlorophenyl)-1H-pyrazole-3-carboxylic acid (I-2d)

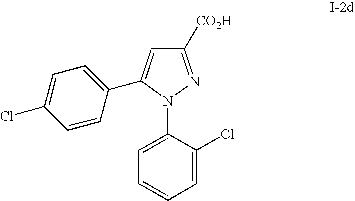

4-(4-Chlorophenyl)-2-hydroxy-4-oxo-but-2-enoic acid ethyl ester lithium salt I-2b (30.26 g, 116 mmoles) was suspended in 242 ml of ethanol. 2-Chlorophenylhydrazine hydrochloride (20.88 g, 116 mmoles) was added portionwise as a solid over 45 minutes while maintaining an internal temperature between 30-40° C. The reaction mixture went from a yellow suspension to a dark orange suspension. The reaction was allowed to stir for 3 hours while maintaining an internal temperature between 25-35° C. An aqueous potassium hydroxide solution (148 ml of 1.8 M solution, 266 mmoles) was added over 20 minutes while maintaining an internal temperature between 20-30° C. The reaction mixture was held for 2.5 hours. Within 30 minutes of potassium hydroxide solution addition, the reaction turned an almost clear, very dark rust orange in color. Aqueous hydrochloric acid (85 ml of 3.9 M solution, 331 mmoles) was added over 15 minutes while maintaining the reaction temperature between 20-30° C. The product precipitated during hydrochloric acid addition. The precipitated product was granulated for 16 hours at room temperature. The crude product was isolated by filtration and the filtercake was washed with 150 ml of water. The filtercake was a yellowish orange solid. After air-drying for 30 minutes, the filtercake was suspended in 480 ml of methanol. The suspension was heated to reflux to give a clear dark orange solution (all solids in solution within 1 hour of reaching reflux) and then held at reflux for 8 hours. The solution was cooled over 4 hours to room temperature, during which time product had precipitated from solution. The reaction mixture was held at room temperature for 10 hours, followed by cooling to 0° C., and stirring for 1.5 hours. Collection of the precipitate by filtration, washing the resulting filtercake with 150 ml of ice-chilled methanol, and drying at 60° C. in vacuo for 3 hours afforded 1-(2-chlorophenyl)-5-(4-chlorophenyl)-1H-pyrazole-3-carboxylic acid I-2d (29.28 g, 76%) as an off-white solid.

¹H-NMR (CD₃CN): δ 7.58-7.45 (m, 4H), 7.31 (d, 2H, J=8.7 Hz), 7.21 (d, 2H, J=8.7 Hz), 7.10 (s, 1H). Mass Spec (ESI): M+1=333.2

### Preparation of 1-(2-chlorophenyl)-5-(4-chlorophenyl)-4-hydroxy-1H-pyrazole-3-carboxylic acid (I-1a)

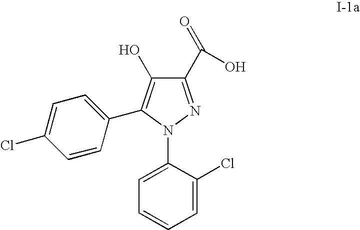

1-(2-Chlorophenyl)-5-(4-chlorophenyl)-1H-pyrazole-3-carboxylic acid I-2d (628.1 g, 1.88 mol) was dissolved in tetrahydrofuran (11 liters) to give a clear light orange solution. This solution was cooled to −78° C. followed by the addition of hexyllithium (2.0 M solution in hexanes, 2.07 liters, 4.14 mol) over a period of 2 hours while keeping internal temperature less than −70° C. During the addition of the first equivalent of hexyllithium, the reaction solution remained clear orange, then during addition of a second equivalent of hexyllithium, the reaction solution turned brown and then very dark green. The reaction mixture was held for 20 minutes at −74° C., then warmed to −50° C. over 30 minutes and held for an additional 1 hour at this temperature. The reaction was cooled back to less than −70° C., followed by the addition of neat trimethylborate (238 g, 2.01 moles) over 3 minutes while keeping the temperature less than −68° C. The reaction solution was then warmed to room temperature over 3 hours. The reaction remained very dark green until reaching room temperature after which it turned clear dark orange. Aqueous sodium hydroxide (750 ml of 3.0 M, 2.25 mol) was added over 5 minutes to crude reaction solution while maintaining an internal temperature of 10-15° C. Concentrated aqueous hydrogen peroxide (253 g, 30 wt %, 2.01 moles) was then added over a period of 30 minutes while maintaining an internal temperature between 10-20° C. The reaction was allowed to warm to room temperature and stirred for 3.5 hours. Water (3 liters) was added followed by addition of concentrated aqueous hydrochloric acid (545 ml, 12.1 M, 6.59 mol) over 15 minutes while maintaining a temperature of 20-30° C. The pH of the crude reaction solution was approximately 2.5. The tetrahydrofuran and aqueous layers were separated and the aqueous layer was extracted with 4 liters of tert-butyl methyl ether. The tetrahydrofuran and tert-butyl methyl ether layers were combined, washed with 4 liters of brine, and dried over 2.5 Kg of Na₂SO₄. The crude solution was concentrated in vacuo to a thick orange oil containing some fine solids. The crude orange oil was then added to 5 liters of methanol, causing a bright yellow precipitate to crystallize from solution. The precipitated product was granulated for 20 hours at room temperature followed by cooling to 0° C. and stirring for 1 hour. The crude product was isolated by filtration and the resulting filtercake was washed with 1 liter of ice-chilled methanol. The filtercake was air-dried for 18 hours. This crude product (390 g) was suspended in 2.1 liters of 2-propanol followed by heating to reflux to give a clear yellow/orange solution. Solution held at reflux for 1 hour, then cooled over a period of 5 hours to 3° C. and stirred for 1 hour. The recrystallized product was isolated by filtration and the resulting filtercake was washed with 900 ml of ice-chilled 2-propanol, followed by air-drying for 18 hours. The product was oven-dried for 18 hours at 60° C. and 10 mm to afford 1-(2-chlorophenyl)-5-(4-chlorophenyl)-4-hydroxy-1H-pyrazole-3-carboxylic acid I-1a (282.9 g, 43%) as an off-white solid.

¹H-NMR (CD₃CN): δ 7.55-7.44 (m, 4H), 7.31 (d, 2H, J=8.7 Hz), 7.20 (d, 2H, J=8.7 Hz). Mass Spec (ESI): M+1=349.2

### Preparation of Starting material 2-(2,2-Difluoropropylamino)-ethanol (Sm-1c)

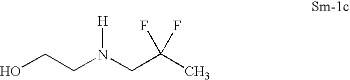

Two 22 liter 3-neck round-bottomed flasks equipped with a mechanical stirrer, thermometer, nitrogen inlet and drying tubes were each charged with ethyl pyruvate (2350 g, 20.24 mol) and then cooled to −15 to −10° C. using a dry ice/acetone bath. [Bis(2-methoxyethyl)amino]sulfur trifluoride (DeoxoFluor™; 3731 ml, 20.24 mol) was then added to each of the flasks while maintaining the temperature less than −5° C. The reaction mixtures were then stirred at a temperature less than 30° C. until no more starting material was present by gas chromatography (GC). Each reaction mixture was then added to a stirred mixture of water (16.6 Kg)/ice(16.6 Kg) and sodium bicarbonate (3.3 Kg) in a 30 gallon crock over approximately 30 minutes. The aqueous mixtures were then stirred overnight to room temperature. The two layers were separated for each crude reaction mixture and the aqueous layers were split in half and each extracted with methylene chloride (3× 2 liters). The methylene chloride layers were combined (24 L total) and then washed with brine (1× 2 liters) followed by treatment with charcoal and dried over magnesium sulfate. The solvent was removed and the liquid residue distilled at 60-110° C. at atmospheric pressure to yield 5557 g (99.4%) of 2,2-difluoro-propionic acid ethyl ester.

A 12 liter 3-necked round-bottomed flask equipped with a mechanical stirrer, thermometer, addition funnel, condenser, nitrogen inlet and drying tube was charged with 2,2-difluoro propionic acid ethyl ester from above (5557 g) and heated to reflux (approximately 53° C.). Ethanolamine (2457 g, 4023 mol) was added to the heated solution over approximately 1 hour while maintaining a gentle reflux. After the addition was complete, the mixture was refluxed for an additional hour (reaction was deemed complete by GC analysis). Ethanol was removed from the reaction mixture by vacuum distillation. The crude product was then crystallized by diluting with toluene (1:1) and followed by cooling to −20° C. After solids started to precipitate out of solution, hexane (4 ml/1 ml) was added and the mixture was allowed to stir for 2 additional hours at −20° C., followed by holding the mixture in a freezer overnight. The solids were filtered through a polypad and washed with freezer cold hexane (2× 1 liter). The isolated solids were then dried in vacuo with no heat to give 3000 g (49%) of 2,2-difluoro-N-(2-hydroxyethyl)-propionamide as a low melting solid (mp=36-38° C.).

Two 50 liter 3-necked round-bottomed flasks were each equipped with a mechanical stirrer, thermometer, addition funnel, nitrogen inlet and a drying tube. Tetrahydrofuran (THF: 9 liters) was charged to each flask and then cooled to −5° C. Lithium aluminum hydride (734.5 g, 31.18 mol) was added to each reaction flask portionwise while monitoring gas evolution and maintaining the temperature less than 25° C. using an ice/methanol bath. After the additions were complete, the reaction mixture was cooled to 0° C. and 2,2-difluoro-N-(2-hydroxyethyl)propanamide from above (1500 g, 9.79 mol) dissolved in THF (9 liters) was added to each reaction mixture while maintaining the temperature less than 30° C. After about 48-60 hours, the mixtures were cooled to 0° C. using an ice/methanol bath and quenched with a 10% sodium hydroxide solution (3.2 liters) while maintaining the temperature between 0-30° C. The reaction mixtures were filtered through an 18 inch (45.72 cm) crock funnel using a polypad. The solids from each reaction were slurried with methylene chloride (3× 7 liters) and filtered. All of the filtrates were then combined and concentrated to dryness in vacuo. The residue was distilled and the product collected between 95-100° C. at 25 mm Hg. All of the material collected was redistilled and the product (2-[(2,2-difluoropropyl)amino]ethan-1-ol Sm-1c was collected between 85° C./17 mm Hg and 102° C./25 mm Hg.

The following describes an alternative procedure for the preparation of Intermediate 5-(4-chlorophenyl)-1-(2-chlorophenyl)-4-hydroxy-1H-pyrazole-3-carboxylic Acid (I-1a).

### Preparation of Intermediate 4-(4-Chlorophenyl)-3-oxo-butyric Acid Ethyl Ester (I-3a)

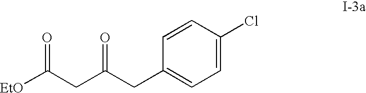

Pyridine (105 ml) was added dropwise over a 30-minute period o a cooled (0° C.) to a stirred solution of 2,2-dimethyl-1,3-doxane-4,6-dione (78.5 g, 0.54 mol) in dichloromethane (200 ml). A solution of 4-chlorophenylacetyl chloride (100 g, 0.53 mol) in dichloromethane (150 ml) was then added dropwise. The reaction mixture was stirred for 1 hour at 0° C., The cooling bath was removed, and stirring was continued for an additional 2 hours. The reaction mixture was poured over 2N hydrochloric acid (aq.)/ice, layers separated and the aqueous layer washed with dichloromethane (2×150 ml). Combined organic layers were washed with 2N hydrochloric acid (aq.) (2×150 ml), brine, dried (Na₂SO₄) and concentrated in vacuo to afford a solid.

The material obtained above was slurried in ethanol (1 liter), heated to reflux for 3 hours, then cooled and concentrated in vacuo. The oily residue was fractionally-distilled under vacuum to afford the title compound (I-3a) as a clear oil, 108 g.

### Preparation of Intermediate 4-(4-Chlorophenyl)-2-[(2-chlorophenyl)-hydrazono]-3-oxobutyric Acid Ethyl Ester (I-3b)

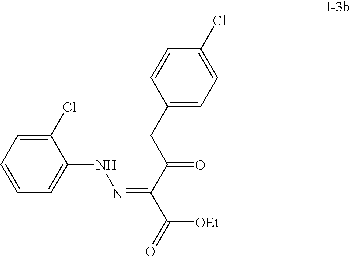

A solution of sodium nitrite (3.4 g, 50.4 mmol) in water (15 ml) was added dropwise over an hour period to a cooled (0° C.), stirred solution of 2-chloroaniline (6.4 g, 50.4 mmol) in acetic acid (50 ml)/water (7 ml). Then a solution of 4-(4-chloro-phenyl)-3-oxo-butyric acid ethyl ester I-3a (10 g, 42 mmol) in acetic acid (30 ml) was added dropwise over a 30-minutes period to produce an orange slurry (20 ml of water added to aid stirring). After an additional hour, the mixture was filtered, solids washed with water and air-dried. Solids slurried in ethanol (75 ml) for 30 minutes, filtered, solids washed with methanol and dried in vacuo to afford the title compound (I-3b) as an orange solid, 11.0 g.

### Preparation of 4-Bromo-4-(4-chlorophenyl)-2-[(2-chlorophenyl)-hydrazono]-3-oxobutyric Acid Ethyl Ester (I-3c)

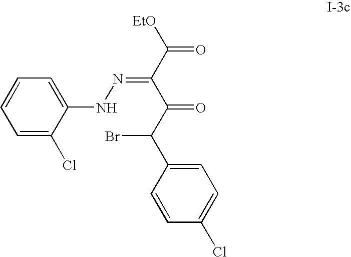

A stirred slurry of 4-(4-chlorophenyl)-2-[(2-chlorophenyl)-hydrazono]-3-oxobutyric acid ethyl ester I-3b (10.0 g, 26 mmol) and copper (II) bromide (13.4 g, 59.8 mmol) in ethyl acetate (100 ml)/chloroform (100 ml) was heated at 60° C. for 3 hours. The reaction mixture was cooled and filtered through diatomaceous earth followed by washing with chloroform. The filtrate was diluted with dichloromethane, washed with water, brine, dried (Na₂SO₄) and concentrated in vacuo to afford the title compound (I-3c) as a red oil, 12.1 g.

### Preparation of intermediate 5-(4-Chlorophenyl)-1-(2-chlorophenyl)-4-hydroxy-1H-pyrazole-3-carboxylic Acid Ethyl Ester (I-3d)

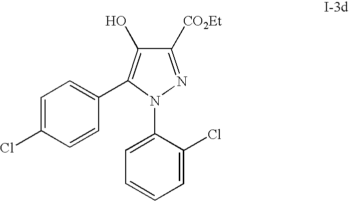

A mixture of 4-bromo-4-(4-chloro-phenyl)-2-[(2-chloro-phenyl)-hydrazono]-3-oxo-butyric acid ethyl ester I-3c (12.1 g, 26 mmol) and sodium acetate (10.8 g, 130 mmol) in methanol (100 ml) was heated at reflux for 4 hours, cooled, and then concentrated in vacuo. The residue was partitioned between ethyl acetate and water. The organic layer was washed with brine, dried (Na₂SO₄) and concentrated in vacuo to afford a solid. A slurry of this material in cyclohexane was heated to reflux and allowed to stir at ambient temperature for 2 hours and then filtered to afford the title compound (I-3d) as a yellow solid (I-1d), 6.5 g.

### Preparation of Intermediate 5-(4-chlorophenyl)-1-(2-chlorophenyl)-4-hydroxy-1H-pyrazole-3-carboxylic Acid (I-1a)

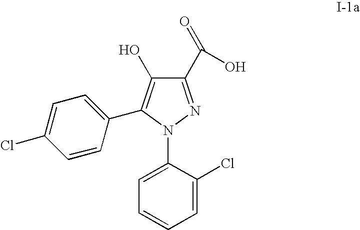

Aqueous potassium hydroxide (200 ml, 3.18 M, 636 mmol) was diluted with 1 liter of methanol followed by portionwise addition of 1-(2-chlorophenyl)-5-(4-chlorophenyl)-4-hydroxy-1H-pyrazole-3-carboxylic acid ethyl ester I-3d (100 g, 266 mmol) as a solid. Initially, a clear dark orange solution formed, but solids quickly precipitated back out of solution. The reaction mixture was then heated to reflux (a clear dark orange solution was obtained at 55° C.). The reaction was held at reflux (70° C.) for 4 hours, followed by cooling to room temperature (a small amount of precipitated out of solution). Another 1 liter of methanol was added (more precipitate formed) followed by 260 ml of water (a clear dark orange solution). Concentrated aqueous hydrochloric acid was added (57 ml, 12.1 M, 690 mmol) over 10 minutes, keeping the temperature between 20-30° C. (pH˜3). A precipitate began to come out of solution after HCl addition was 70% complete. The mixture was stirred for 1.5 hours at room temperature, then the precipitate was collected by filtration, and the resulting filtercake washed with 500 ml of room temperature 1:1, methanol:water, followed by washing with 500 ml of water. Collected solids were air-dried for 2 hours followed by drying at 60° C. and 1 mm for 15 hours to afford 1-(2-chlorophenyl)-5-(4-chlorophenyl)-4-hydroxy-1H-pyrazole-3-carboxylic acid I-1a (90.8 g, 98%) as an off-white solid.

### Example 1

### Preparation of 3-(4-Chloro-phenyl)-2-(2-chloro-phenyl)-7-(2,2-difluoro-propyl)-6,7-dihydro-2H,5H-4-oxa-1,2,7-triaza-azulen-8-one (1A-1)

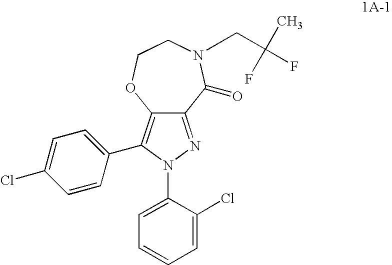

### Preparation of 4-Acetoxy-1-(2-chlorophenyl)-5-(4-chlorophenyl)-1H-pyrazole-3-carboxylic acid (I-1b)

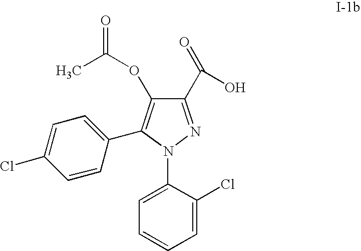

1-(2-Chlorophenyl)-5-(4-chlorophenyl)-4-hydroxy-1H-pyrazole-3-carboxylic acid I-1a (572.0 g, 1.64 mol) was combined with 8 liters of methylene chloride to give an off-white suspension. N,N-Diisopropylethylamine (427.9 g, 3.29 mol) was added over 15 minutes while keeping the temperature between 20-25° C. A clear yellow solution resulted. Acetic anhydride (334.5 g, 3.24 mol) was added over 5 minutes keeping the temperature between 20-25° C. The reaction was stirred at room temperature for 16 hours. The crude reaction solution was washed twice with 4 liter portions of 0.5 M citric acid and once with 4 liters of brine. The crude solution was concentrated in vacuo to a total volume of 1 liter. This milky suspension was then added to 4 liters of hexanes causing the desired product to precipitate instantly. The solids were granulated for 30 minutes and then collected by filtration. The filtercake was rinsed with 3 liters of hexanes and then air-dried for 16 hours. The isolated product was then further dried at 60° C. and 8 mm for 2 hours. 4-Acetoxy-1-(2-chlorophenyl)-5-(4-chlorophenyl)-1H-pyrazole-3-carboxylic acid I-1b (601.6 g, 94%) was isolated as a powdery, off-white solid.

¹H-NMR (CD₂Cl₂): δ 7.50 (d, 1H, J=7.0 Hz), 7.47-7.37 (m, 3H), 7.28 (d, 2H, J=8.7 Hz), 7.12 (d, 2H, J=8.7 Hz), 2.26 (s, 3H). Mass Spec (ESI): M+1=391.2

### Preparation of Acetic acid 1-(2-chlorophenyl)-5-(4-chlorophenyl)-3-[(2,2-difluoro-propyl)-(2-hydroxyethyl)-carbamoyl]-1H-pyrazol-4-yl ester (I-1d)

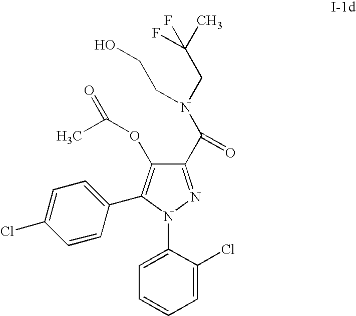

4-Acetoxy-1-(2-chlorophenyl)-5-(4-chlorophenyl)-1H-pyrazole-3-carboxylic acid I-1b (581.0 g, 1.48 mol) was dissolved in 10 liters of methylene chloride to give a pale yellow, slightly opaque solution. The solution was filtered through Celite® to give a clear green colored solution. 2-Chloro-4,6-dimethoxy-1,3,5-triazine (296.8 g, 1.64 mol) was added in one portion as a solid at room temperature to give an opaque suspension (addition is slightly endothermic). 4-Methylmorpholine (182.9 g, 1.80 mol) was added over 15 minutes while keeping the temperature between 18-22° C. (reaction returned to being yellow in color). The reaction was allowed to stir for 3 hours at room temperature, then 2-(2,2-difluoropropylamino)-ethanol Sm-1c (228.3 g, 1.64 mol) was added neat over 10 minutes while keeping the temperature between 20-25° C. The reaction mixture was stirred for 15 hours, then washed twice with 6 liter portions of 10% citric acid and once with 5 liters of brine. The crude product solution was concentrated in vacuo to a thick orange oil and then reconstituted in 4 liters of isopropyl ether. After removing 1 liter of distillates, precipitate began to form. Isopropyl ether (1.5 liters) was added to the crude product suspension and then the mixture was stirred at room temperature for 1 hour. The precipitated solids were collected by filtration and the resulting filtercake was rinsed with 2 liters of room temperature isopropyl ether, followed by air-drying for 16 hours. Acetic acid 1-(2-chlorophenyl)-5-(4-chlorophenyl)-3-[(2,2-difluoro-propyl)-(2-hydroxyethyl)-carbamoyl]-1H-pyrazol-4-yl ester I-1d (603.0 g, 78%) was isolated as a granular off-white solid.

¹H-NMR (CD₂Cl₂): δ 7.50-7.31 (m, 4H), 7.28 (d, 2H, J=8.3 Hz), 7.14 (d, 2H, J=8.7 Hz), 4.41-3.41 (m, various rotamers, 7H), 2.21 (s, 3H), 1.65 (t, 3H, J_{HF}=19.5 Hz).

Mass Spec (ESI): M+1=512.2

### Preparation of Acetic acid 3-[(2-chloroethyl)-(2,2-difluoropropyl)-carbamoyl]-1-(2-chlorophenyl)-5-(4-chlorophenyl)-1H-pyrazol-4-yl-ester (I-1e)

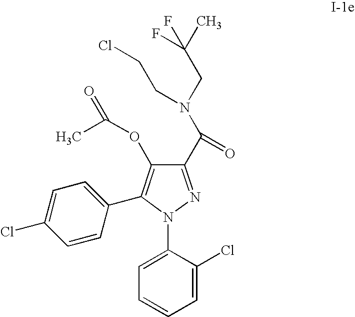

Method A: Acetic acid 1-(2-chlorophenyl)-5-(4-chlorophenyl)-3-[(2,2-difluoro-propyl)-(2-hydroxy-ethyl)-carbamoyl]-1H-pyrazol-4-yl ester I-1d (580.6 g, 1.12 mol) was dissolved in 10 liters of methylene chloride to give a clear pale yellow solution. After cooling to 0° C., methanesulfonyl chloride (142.4 g, 1.21 mol) was added neat over 5 minutes, followed by addition of neat N,N-diisopropylethylamine (167.9 g, 1.29 mol) over 25 minutes, while keeping the temperature less than 5° C. After stirring 20 minutes at <5° C., the reaction was warmed to room temperature and stirred for 14 hours. The crude reaction solution was washed twice with 4.5 liter portions of 10% citric acid and once with 4 liters of brine. The crude product solution was concentrated in vacuo to give a crude solid, then 2 liters of methanol was added followed by stirring for 1 hour. About half of the crude solid had dissolved in and then crystallized from the methanol. This material was collected by filtration and the resulting filtercake was rinsed with 300 ml of room temperature methanol. This first crop of material was dried at 50° C. and 10 mm for 2 hours to give 245.2 g, 41.2% of the title compound as an off-white solid. The crude solid that had not dissolved in and crystallized from methanol was redissolved in 1 liter of methylene chloride, then concentrated to a viscous brownish oil. The methanol mother liquor left over from the first crop was concentrated to a total volume of 800 ml and was then combined with the viscous brownish oil. This mixture was warmed in a 40° C. waterbath until a clear solution was obtained, then the resulting solution was cooled to 0° C. and stirred for 30 minutes, resulting in product precipitation. The precipitate was collected by filtration, and the resulting filtercake was washed with 200 ml of ice-chilled methanol, followed by air-drying for 16 hours. The second crop material (290.9 g, 48.8%) was isolated as an off-white solid. The overall combined yield of first and second crops of acetic acid 3-[(2-chloroethyl)-(2,2-difluoropropyl)-carbamoyl]-1-(2-chlorophenyl)-5-(4-chlorophenyl)-1H-pyrazol-4-yl-ester I-1e was 536.1 g (90%).

Method B: 1-(2-Chlorophenyl)-5-(4-chlorophenyl)-1H-pyrazole-3-carboxylic acid I-2c (90.8 g, 260 mmol) was dissolved in 1.7 liters of methylene chloride, giving an off-white suspension. 4-Methylmorpholine (58.5 g, 576 mmol) was added, giving a clear yellow solution, followed by addition of acetyl chloride (22.6 g, 284 mmol) over 10 minutes while maintaining a temperature between 20-30° C. The reaction was stirred for 7 hours at room temperature, then cooled to 0° C. 2-(2,2-Difluoropropylamino)-ethanol (39.8 g, 286 mmol) was added neat over 1 minute followed by addition of 2-Chloro-4,6-dimethoxy-1,3,5-triazine (49.0 g, 271 mmol) portionwise as a solid over 1 minute. The reaction was allowed to slowly warm to room temperature over a period of 5 hours, followed by stirring for 12 hours at room temperature. The crude reaction solution was washed twice with 900 ml portions of 0.5 M citric acid and once with 900 ml of brine. Residual water was azeotropically removed through two cycles concentrating off methylene chloride and then adding more methylene chloride. The final crude methylene chloride solution volume was 1.2 liters. This solution was cooled to −2° C. followed by addition of neat methanesulfonyl chloride (36.0 g, 311 mmol) and then addition of neat N,N-diisopropylethylamine (42.1 g, 324 mmol) over a 10 minute period while maintaining a reaction temperature less than 10° C. The reaction solution was warmed to room temperature over 1 hour, followed by stirring for 20 hours, then washing the methylene chloride solution twice with 800 ml portions of 0.5 M citric acid and once with 800 ml brine. Product rich methylene chloride layer was clear dark orange in appearance (˜1.3 liters total volume). Crude solution was concentrated in vacuo to ˜300 ml, followed by addition of 1 liter of methanol. Resulting solution was concentrated in vacuo in a 30° C. waterbath by removing 900 ml of distillates. Another 800 ml portion of methanol was added followed by a final concentration in vacuo (30° C. waterbath) to remove 700 ml of distillates. The final total volume was ˜500 ml. The product rich concentrated solution was held at room temperature for 1 hour (solution was initially hazy, dark orange in appearance, then solids precipitated after ˜15 minutes). The mixture was cooled to −10° C. and stirred for 1 hour while maintaining temperature less than 0° C. The precipitated solids were collected by filtration, and the resulting filtercake was washed with 50 ml of ice-chilled methanol, followed by air-drying for 15 hours. The isolated solids were further dried at 60° C. and 1 mm for 2 hours (loss on drying was only 0.4 g) to give acetic acid 3-[(2-chloroethyl)-(2,2-difluoropropyl)-carbamoyl]-1-(2-chlorophenyl)-5-(4-chlorophenyl)-1H-pyrazol-4-yl-ester I-1e (104.0 g, 75%) as a white solid.

¹H-NMR (CD₂Cl₂): δ 7.49-7.47 (m, 1H), 7.44-7.40 (m, 1H), 7.37-7.33 (m, 2H), 7.28 (d, 2H, rotamers, J=8.7 Hz), 7.14 (d, 2H, rotamers, J=8.7 Hz), 4.46 (t, 0.72H, J cannot be determined), 4.14 (t, 1.28H, J cannot be determined), 3.97 (t, 1.28H, J_{HF}=13.0 Hz), 3.87 (t, 0.72H, J=6.4 Hz), 2.22 (s, 1.08H), 2.20 (s, 1.92H), 1.62 (t, 3H, rotamers, J_{HF}=19.5 Hz). Two major rotamers present in a ˜1.7:1 ratio.

Mass Spec (ESI): M+1=530.2

### Preparation of 1-(2-chlorophenyl)-5-(4-chlorophenyl)-4-hydroxy-1H-pyrazole-3-carboxylic acid (2-chloroethyl)-(2,2-difluoropropyl)-amide (I-1f)

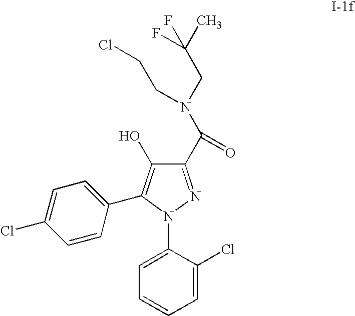

Acetic acid 3-[(2-chloroethyl)-(2,2-difluoropropyl)-carbamoyl]-1-(2-chlorophenyl)-5-(4-chlorophenyl)-1-H-pyrazol-4-yl-ester I-1e (3.55 g, 6.69 mmol) was dissolved in 90 ml of methanol with warming in a 40° C. waterbath to give a clear colorless solution. The resulting solution was cooled to 0° C. (still a clear, colorless solution), followed by addition of K₂CO₃ (1.02 g, 7.31 mmol) in one portion as a solid (reaction mixture goes from colorless to yellow). The reaction was stirred for 30 minutes at 0° C. followed by addition of concentrated hydrochloric acid (1.2 ml of 12.1 M, 14.5 mmol). Upon neutralization, the reaction turned colorless and clear, then product began to precipitate. The reaction was warmed to room temperature, then 45 ml of water was added, followed by stirring for 2.5 hours. The precipitated solids were collected by filtration and the resulting filtercake was washed with 50 ml of room temperature 2:1, methanol:water. The collected solids were dried at 50° C. in vacuo for 1 hour to give 1-(2-chlorophenyl)-5-(4-chlorophenyl)-4-hydroxy-1H-pyrazole-3-carboxylic acid (2-chloroethyl)-(2,2-difluoropropyl)-amide I-1f (2.86 g, 87%) as a white solid.

¹H-NMR (CD₂Cl₂): δ 9.67 (s, 0.52H), 9.57 (s, 0.48H), 7.51-7.48 (m, 1H), 7.46-7.41 (m, 1H), 7.39-7.31 (m, 2H), 7.24 (d, 2H, rotamers, J=8.7 Hz), 7.17 (d, 2H, rotamers, J=8.7 Hz), 4.75 (t, 0.52H, J_{HF}=13 Hz), 4.47 (t, 0.48H, J=6 Hz), 4.08 (t, 0.48H, J_{HF}=13 Hz), 3.94 (t, 0.52H, J=6 Hz), 3.84-3.79 (m, 2H), 1.66 (t, 1.44H, J_{HF}=19.3 Hz), 1.59 (t, 1.56H, J_{HF}=19.1 Hz). Two major rotamers present in a ˜1.07:1 ratio.

Mass Spec (ESI): M+1=488.2

### Preparation of 3-(4-Chlorophenyl)-2-(2-chlorophenyl)-7-(2,2-difluoropropyl)-6,7-dihydro-2H,5H-4-oxa-1,2,7-triaza-azulen-8-one (I-1A)

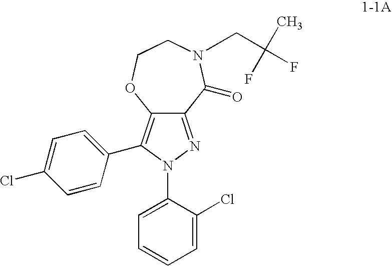

Acetic acid 3-[(2-chloroethyl)-(2,2-difluoropropyl)-carbamoyl]-1-(2-chlorophenyl)-5-(4-chlorophenyl)-1H-pyrazol-4-yl-ester I-1f (513.0 g, 0.97 mol) was suspended in 9.7 liter of ethanol (a off-white suspension). Cesium carbonate (348.0 g, 1.07 mol) was added portionwise as a solid over 2 minutes while maintaining an internal temperature between 21-27° C. Upon Cs₂CO₃ addition, the reaction mixture turned pale yellow (still a suspension). The reaction was allowed to stir at room temperature for 19 hours, then the crude reaction mixture was filtered through Celite® to remove insoluble solids, giving a clear dark yellow filtrate. The Celite® filtercake was washed with 2 liters of ethanol. The crude product solution was concentrated in vacuo and gave a yellow solid. This solid was reconstituted in 7 liters of methylene chloride and the resulting mixture was washed once with 5 liters of half saturated aqueous NH₄Cl and once with 4 liters of brine. The product rich methylene chloride layer was concentrated in vacuo to a total volume of 2.5 liters. The methylene chloride layer was clear and dark reddish in color. The product rich methylene chloride solution was treated with 105 g of Darco, followed by stirring at reflux for 30 minutes. After cooling, the Darco was filtered off by passing the solution through Celite®. The crude product solution was clear dark orange in appearance. The crude product filtrate was concentrated in vacuo to a total volume of 1.1 liters. This product rich methylene chloride solution was added over 20 minutes to 5 liters of cyclohexane while maintaining a reaction pot temperature of 50-60° C. Halfway through the methylene chloride solution addition, precipitate came out of solution. After complete addition, the methylene chloride solvent was removed at atmospheric pressure (3.55 liters of distillates collected while simultaneously adding 2 liters of cyclohexane to refluxing solution) from the reaction mixture by heating to 79° C. (internal pot temperature) over a 2.5 hour period. Once the internal temperature reached the boiling point of cyclohexane, all of the methylene chloride had been displaced. The reaction mixture took on a very dark pink/purple coloration with white solids suspended. The reaction mixture was held at 79° C. for 10 minutes, cooled to 50° C. and then held for 13 hours, followed by cooling to 30° C. and holding for an additional 4 hours. The precipitated product was collected by filtration, and the resulting filtercake was washed with 3 liters of room temperature cyclohexane, followed by air-drying for 3.5 hours. The isolated solids were further dried at 50° C. and 2 mm for 15 hours (loss on drying was only 0.2 g) to give 3-(4-chlorophenyl)-2-(2-chlorophenyl)-7-(2,2-difluoropropyl)-6,7-dihydro-2H,5H-4-oxa-1,2,7-triaza-azulen-8-one 1-1A (321.3 g, 73%) as an off-white solid.

### Recrystallization of 3-(4-Chlorophenyl)-2-(2-chlorophenyl)-7-(2,2-difluoropropyl)-6,7-dihydro-2H,5H-4-oxa-1,2,7-triaza-azulen-8-one (1-1A)

3-(4-Chlorophenyl)-2-(2-chlorophenyl)-7-(2,2-difluoropropyl)-6,7-dihydro-2H,5H-4-oxa-1,2,7-triaza-azulen-8-one 1-1A (5.00 g, 11.1 mmol) was dissolved in 20 ml of methylene chloride to give a clear orange solution. Darco KBB (0.5 g) was added followed by heating to reflux and stirring for 1 hour. After cooling, the Darco KBB was filtered off by passing the solution through Celite®, giving a clear light yellow filtrate. The Celite® filtercake was washed with 10 ml of methylene chloride. The eluent was concentrated in vacuo to give a total solution volume of ˜20 ml. The concentrated methylene chloride solution was then diluted with 150 ml of 2-propanol to give a clear pale yellow solution. Methylene chloride was removed from the resulting solution by atmospherically distilling off 71 ml of distillates as solution was heated from room temperature to 82° C. (boiling point of 2-propanol). The solution was then cooled over 3 hours from 82° C. to room temperature. Note: Solution became hazy around 34° C., followed by precipitate formation. The mixture was stirred at room temperature for 62 hours, then cooled to 0° C. and stirred for 2.5 hours before collecting the precipitate by filtration. The resulting filtercake was washed with 80 ml of ice-chilled 2-propanol, then air-dried for 1 hour. The recrystallized 3-(4-Chlorophenyl)-2-(2-chlorophenyl)-7-(2,2-difluoropropyl)-6,7-dihydro-2H,5H-4-oxa-1,2,7-triaza-azulen-8-one 1-1A (4.03 g, 81%) was isolated as a pure white crystalline solid.

¹H-NMR (CD₂Cl₂): δ 7.49-7.46 (m, 1H), 7.45-7.37 (m, 3H), 7.24 (d, 2H, rotamers, J=9.1 Hz), 7.16 (d, 2H, rotamers, J=8.7 Hz), 4.44 (dd, 2H, J=5.2 Hz, 1.9 Hz), 3.98 (t, 2H, J_{HF}=13 Hz), 3.87 (t, 2H, J=3.7 Hz), 1.67 (t, 3H, J_{HF}=19.1 Hz)

Mass Spec (ESI): M+1=452.2

What is claimed is:

1. A process for preparing a compound having the Formula (I) 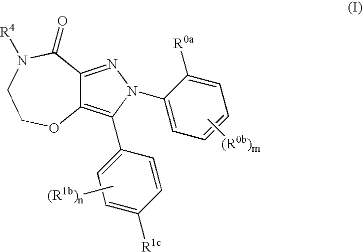
wherein
R^{0a}, R^{0b}, R^{1b}, and R^{1c} are each independently halo, (C₁-C₄)alkoxy, (C₁-C₄)alkyl, halo-substituted (C₁-C₄)alkyl, or cyano;
n and m are each independently 0, 1 or 2; and
R⁴ is a chemical moiety selected from the group consisting of (C₁-C₈)alkyl, aryl, heteroaryl, aryl(C₁-C₄)alkyl, a 3- to 8-membered partially or fully saturated carbocyclic ring(s), heteroaryl(C₁-C₃)alkyl, 5-6 membered lactone, 5- to 6-membered lactam, and a 3- to 8-membered partially or fully saturated heterocycle, where said chemical moiety is optionally substituted with one or more substituents;
a pharmaceutically acceptable salt thereof, or a solvate or hydrate of said compound, or said salt;
comprising the steps of:
(1) protecting the hydroxy group of a compound of Formula (1a) with a hydroxy-protecting group to form a hydroxy-protected compound of Formula (1b) 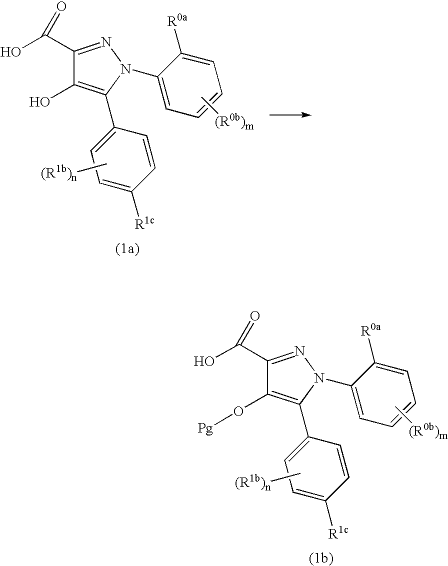
where R^{0a}, R^{0b}, R^{1b}, R^{1c}, n and m are as defined above, and Pg is a hydroxy-protecting group;
(2) reacting said hydroxy-protected compound of Formula (1b) with a compound of Formula (1c) to form a compound of Formula (1d) 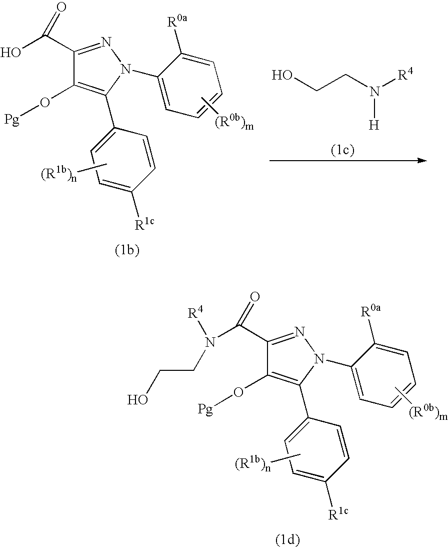
where R^{0a}, R^{0b}, R^{1b}, R^{1c}, n, m, Pg, and R⁴ are as defined above;
(3) converting the hydroxy group of said compound of Formula (1d) to a leaving group to produce a compound of Formula (1e) 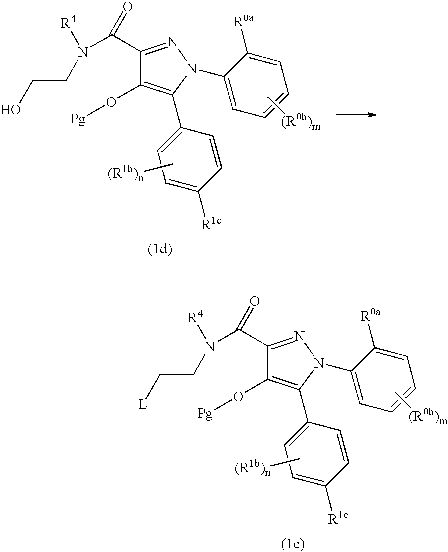
where R^{0a}, R^{0b}, R^{1b}, R^{1c}, n, m, Pg, and R⁴ are as defined above and L is a leaving group;
(4) removing said hydroxy-protecting group and cyclizing said compound of Formula (1e) to form the compound of Formula (I); and
(5) isolating said compound of Formula (I) or a pharmaceutically acceptable salt thereof, or a solvate or hydrate of said compound, or said salt.

2. The process of claim 1 wherein said compound of Formula (1a) is converted to said compound of Formula (1e) without isolating, said compound of Formula (1b) or said compound of Formula (1d).

3. The process of claim 1 wherein said compound of Formula (1a) is prepared by a method comprising the steps of
(i) reacting a compound of Formula (2a) with dialkyl oxalate in the presence of an alkali metal base to form a compound of Formula (2b) 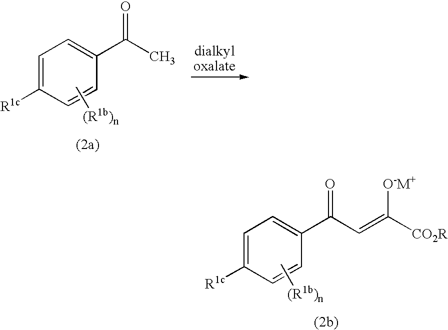
where R^{1b} and R^{1c} are each independently halo, (C₁-C₄)alkoxy, (C₁-C₄)alkyl, halo-substituted (C₁-C₄)alkyl, or cyano, n is 0, 1 or 2; M is an alkali metal and R is (C₁-C₆)alkyl;
(ii) reacting said compound of Formula (2b) with a compound of Formula (2c) to form a compound of Formula (2d) 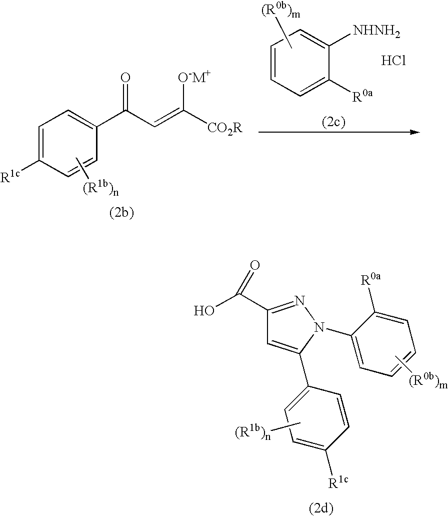
where R^{1b}, R^{1c}, n, and M are as defined above; R^{0a} and R^{0b} are each independently halo, (C₁-C₄)alkoxy, (C₁-C₄)alkyl, halo-substituted (C₁-C₄)alkyl, or cyano, m is 0, 1 or 2 and R is (C₁-C₆)alkyl; and
(iii) reacting said compound of Formula (2d) with an (C₁-C₆)alkyl lithium followed by a trialkylborate and then treating with basic hydrogen peroxide to produce said compound of Formula (1a).

4. The process of claim 1 wherein said compound of Formula (1a) is prepared by a method comprising the step of
(i) hydrolyzing a compound of Formula (3d) to form said compound of Formula (1a) 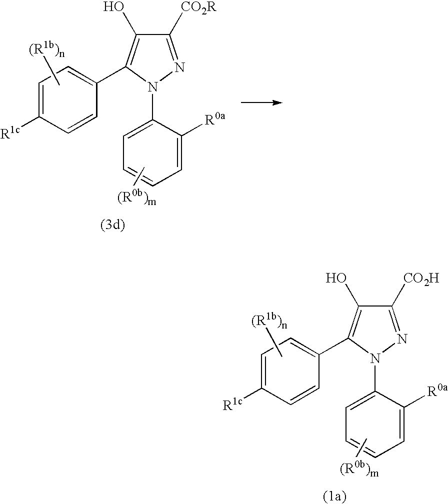
where R^{0a}, R^{0b}, R^{1b}, and R^{1c} are each independently halo, (C₁-C₄)alkoxy, (C₁-C₄)alkyl, halo-substituted (C₁-C₄)alkyl, or cyano; n and m are each independently 0, 1 or 2; and R is (C₁-C₆)alkyl.

5. A process for preparing 3-(4-chloro-phenyl)-2-(2-chloro-phenyl)-7-(2,2-difluoro-propyl)-6,7-dihydro-2H,5H-4-oxa-1,2,7-triaza-azulen-8-one, or a solvate or hydrate thereof;
comprising the steps of:
(1) protecting the hydroxy group of a compound of Formula (1a-1) with an acetyl group to form a compound of Formula (1b-1); 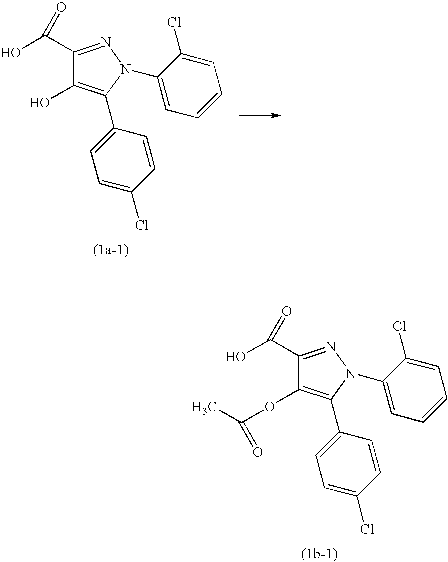
(2) reacting said compound of Formula (1b-1)with a compound of Formula (1c-1) to form a compound of Formula (1d-1) 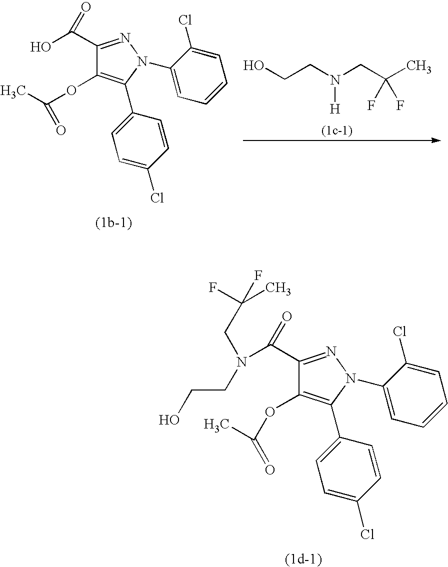
(3) converting the hydroxy group of said compound of Formula (1d-1) to a chloro group to produce a compound of Formula (1e-1) 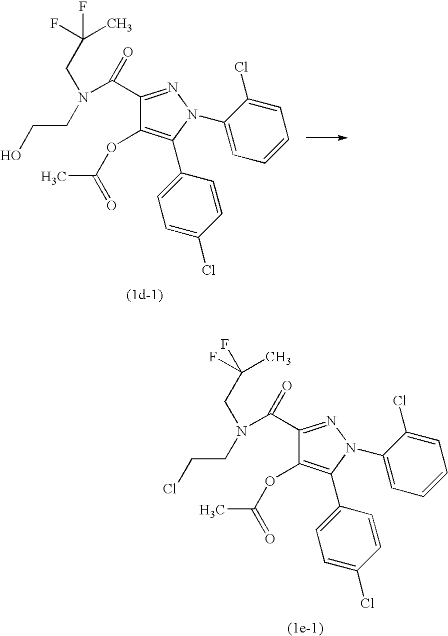
(4) removing said acetyl group and cyclizing said compound of Formula (1e-1) to form 3-(4-chloro-phenyl)-2-(2-chloro-phenyl)-7-(2,2-difluoro-propyl)-6,7-dihydro-2H,5H-4-oxa-1,2,7-triaza-azulen-8-one; and
(5) isolating said 3-(4-chloro-phenyl)-2-(2-chloro-phenyl)-7-(2,2-difluoro-propyl)-6,7-dihydro-2H,5H-oxa-1,2,7-triaza-azulen-8-one, or a solvate or hydrate thereof.

6. The process of claim 5 wherein said compound of Formula (1a-1) is converted to said compound of Formula (1e-1) without isolating said compound of Formula (1b-1) or said compound of Formula (1d-1).

7. The process of claim 5 wherein said compound of Formula (I-1a) is prepared by reacting a compound of Formula (2d-1) with hexyllithium, then trimethylborate, followed by treatment with basic hydrogen peroxide to produce said compound of Formula (1a-1)

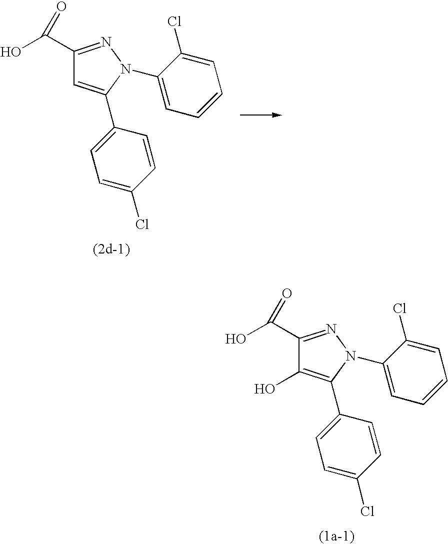

8. A compound having the Formula (1d) 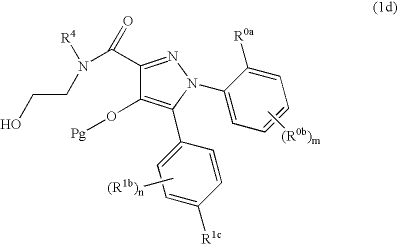
wherein
Pg is a hydroxy-protecting group;
R^{0a}, R^{0b}, R^{1b}, and R^{1c} are each independently halo, (C₁-C₄)alkoxy, (C₁-C₄)alkyl, halo-substituted (C₁-C₄)alkyl, or cyano;
n and m are each independently 0, 1 or 2; and
R⁴ is a chemical moiety selected from the group consisting of (C₁-C₈)alkyl, aryl, heteroaryl, aryl(C₁-C₄)alkyl, a 3- to 8-membered partially or fully saturated carbocyclic ring(s), heteroaryl(C₁-C₃)alkyl, 5-6 membered lactone, 5- to 6-membered lactam, and a 3- to 8-membered partially or fully saturated heterocycle, where said chemical moiety is optionally substituted with one or more substituents.

9. The compound of claim 8 wherein Pg is acetyl; R^{0a} and R^{1c} are both chloro; m and n are both 0; and R⁴ is 2,2-difluoro-n-propyl.

10. A compound having the Formula (1e) 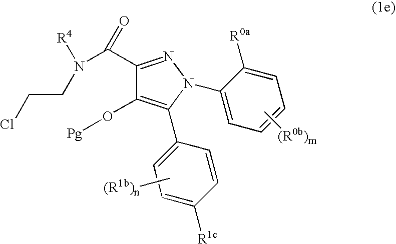
wherein
Pg is a hydroxy-protecting group;
R^{0a}, R^{0b}, R^{1b}, and R^{1c} are each independently halo, (C₁-C₄)alkoxy, (C₁-C₄)alkyl, halo-substituted (C₁-C₄)alkyl, or cyano;
n and m are each independently 0, 1 or 2; and
R⁴ is a chemical moiety selected from the group consisting of (C₁-C₈)alkyl, aryl, heteroaryl, aryl(C₁-C₄)alkyl, a 3- to 8-membered partially or fully saturated carbocyclic ring(s), heteroaryl(C₁-C₃)alkyl, 5-6 membered lactone, 5- to 6-membered lactam, and a 3- to 8-membered partially or fully saturated heterocycle, where said chemical moiety is optionally substituted with one or more substituents.

11. The compound of claim 10 wherein Pg is acetyl; R^{0a} and R^{1c} are both chloro; m and n are both 0; and R⁴ is 2,2-difluoro-n-propyl.
